(19) United States
(12) Patent Application Publication
(10) Pub. No.: US 2024/0268745 A1
(43) Pub. Date: Aug. 15, 2024

(54) ELECTRODE CATHETER, POTENTIAL DETECTION DEVICE, AND POTENTIAL DETECTION METHOD

(71) Applicant: Japan Lifeline Co., Ltd., Tokyo (JP)

(72) Inventors: Kenji MORI, Tokyo (JP); Toshiyuki IIJIMA, Tokyo (JP); Keisuke ADACHI, Tokyo (JP)

(21) Appl. No.: 18/436,204

(22) Filed: Feb. 8, 2024

(30) Foreign Application Priority Data
Feb. 10, 2023 (JP) 2023-018905

(51) Int. Cl. A61B 5/367; A61B 5/00; A61B 5/263; A61B 5/287

(52) U.S. Cl. CPC A61B 5/367; A61B 5/263; A61B 5/287; A61B 5/6858

(57) ABSTRACT

An electrode catheter includes a base material having a thin-film shape, a shape support structure of the base material, and a plurality of electrodes provided on the base material. At least some of the plurality of electrodes are exposed on a front surface and a back surface of the base material. This can improve followability with respect to a dissection.

### CROSS-REFERENCE TO RELATED APPLICATIONS

This application claims the benefit of priority to Japanese Patent Application Number 2023-018905 filed on Feb. 10, 2023. The entire contents of the above-identified application are hereby incorporated by reference.

### TECHNICAL FIELD

The present invention relates to an electrode catheter, a potential detection device, and a potential detection method.

### BACKGROUND

In the related art, an electrode catheter disclosed in JP 6528010 B is known as an electrode catheter for detecting a potential of an organ such as the heart. The electrode catheter of JP 6528010 B includes four arms extending substantially in parallel, each of which is provided with a plurality of electrodes.

### SUMMARY

The electrode catheter disclosed in JP 6528010 B has low shape followability with respect to a dissection having a complex shape, and it is sometimes difficult to cause the electrode to contact the dissection as the operator (therapist) desires.

An object of the present disclosure is to provide an electrode catheter with improved followability with respect to a dissection.

To solve the above problem, one aspect of the present disclosure includes a base material having a thin-film shape, a shape support structure of the base material, and a plurality of electrodes provided on the base material. At least some of the plurality of electrodes are exposed on a front surface and a back surface of the base material.

### BRIEF DESCRIPTION OF DRAWINGS

FIG. 1 is a perspective view of an electrode catheter according to an embodiment.

FIG. 2 is a schematic diagram of a base material of the electrode catheter.

FIG. 3 is a schematic diagram of the base material of the electrode catheter.

FIG. 4 is a schematic diagram of the base material of the electrode catheter.

FIG. 5 is a schematic diagram of the base material of the electrode catheter.

FIG. 6 is a perspective view illustrating an overview of the base material.

FIG. 7 is a perspective view illustrating a shape support structure.

FIG. 8 is a schematic diagram illustrating a shaft.

FIG. 9 is a side view of the electrode catheter.

FIG. 10 is a side view of the electrode catheter.

FIG. 11 is a diagram illustrating a usage state of the electrode catheter.

FIG. 12 is a diagram illustrating a usage state of the electrode catheter.

### DESCRIPTION OF EMBODIMENTS

An embodiment of the present disclosure will be described below. In the embodiment, a so-called mapping electrode catheter is used as an example of a potential detection device. The present disclosure can be used to detect a potential of various objects having a complex surface shape. In the following embodiment, three-dimensional Cartesian coordinates may be used to describe direction. The Z-axis of the three-dimensional Cartesian coordinate is defined to extend in the axial direction of a shaft, with the distal side of the shaft (distal side viewed from an operator) referring to the negative direction and the proximal side of the shaft (proximal side viewed from the operator) referring to the positive direction. The X-axis extends in a direction orthogonal to the axis of the shaft, that is, in the radial direction of the shaft. For the convenience of description, the X-axis extends in the longitudinal direction of a base material (any one of a plurality of base materials) described below. The root side of the base material refers to the negative direction of the X-axis and the tip side of the base material refers to the positive direction of the X-axis. The Y-axis is orthogonal to the X-axis and Z-axis.

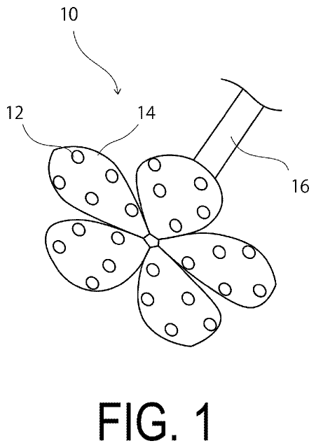

FIG. 1 is a perspective view of the electrode catheter according to the embodiment. As illustrated in FIG. 1, an electrode catheter 10 mainly includes a plurality of electrodes 12, a base material 14, and a shaft 16. As an example, the electrode catheter 10 is a so-called mapping catheter used to obtain a potential at a plurality of locations in the heart and create a three-dimensional map from the obtained results.

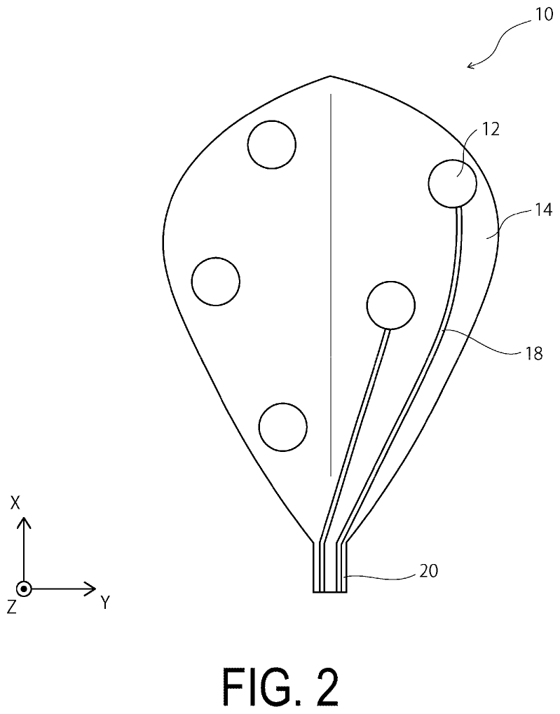

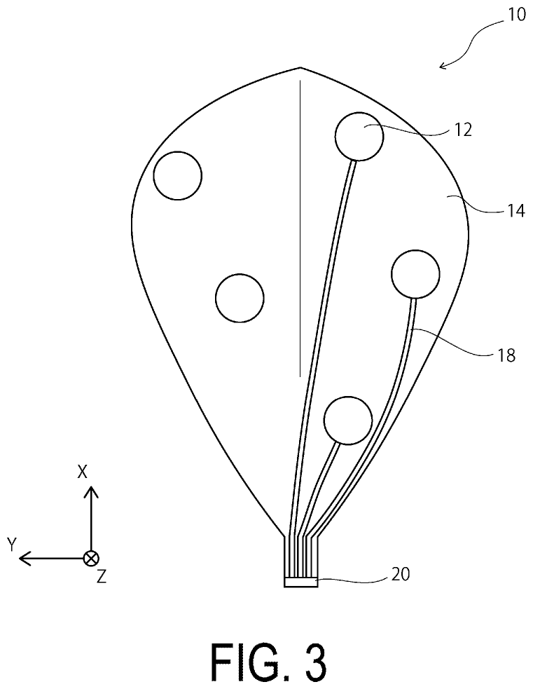

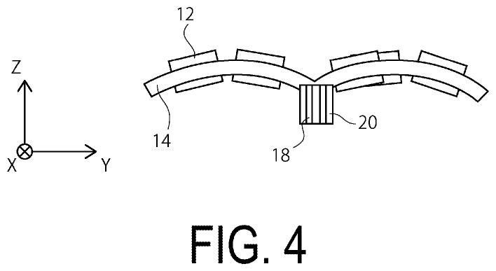

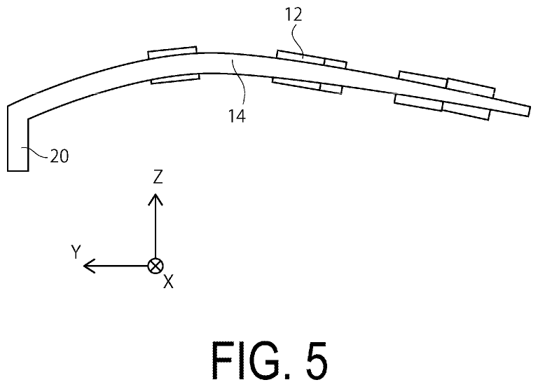

FIGS. 2 to 5 are schematic diagrams of the base material of the electrode catheter. More specifically, FIG. 2 is a view of the base material 14 viewed from one main surface direction (from the front side, i.e., +Z direction), and FIG. 3 is a view of the base material 14 viewed from the other main surface direction (from the back side, i.e., −Z direction). FIG. 4 is a view of the base material 14 viewed from −X direction, and FIG. 5 is a view of the base material 14 viewed from −Y direction. As illustrated in FIGS. 2 to 5, the base material 14 has a shape imitating a so-called lanceolate leaf. More specifically, the base material 14 has a shape in the XY plane in which the width gradually increases from the root side to the tip side and then gradually decreases through an inflection point. The base material 14 has a curved shape in both the XZ and YZ planes. The base material 14 is formed by processing a thin film sheet into a predetermined shape. As a material constituting the base material 14, a nonwoven fabric in which a resin material such as a fibrous polyurethane, polytetrafluoroethylene, nylon, polyethylene terephthalate, polyvinyl alcohol or the like is randomly accumulated can be used. The so-called electrospinning technique can be used as the fiber integration method. In other words, it can be said that the base material 14 is a nonwoven fabric sheet. Any base material 14 may be used as long as the base material 14 has a thin-film shape and the material constituting an electrode 12 can be impregnated. Thus, a woven fabric may be used as the base material 14.

The plurality of electrodes 12 (5 electrodes in the illustrated example) are provided on the base material 14. Each electrode 12 is provided to be exposed on both sides of the base material 14. That is, when the base material 14 is viewed from the front surface and from the back surface, the plurality of electrodes 12 are provided at corresponding positions. The electrodes 12 in the corresponding position may be an integral piece formed so that one electrode 12 penetrates the base material 14, or may be two independent electrodes 12 that are electrically connected to each other. When the electrode 12 is formed to penetrate the base material 14, the electrode 12 may be formed by impregnating the base material 14 with conductive ink. In order to form the electrode 12 by impregnating the base material 14 with conductive ink, a cloth (nonwoven fabric or woven fabric) may be used as the base material 14. The number of electrodes 12 can be appropriately selected according to the application and the part to be applied. As an example, the number of electrodes 12 (total number of electrodes 12) may be 20 or more.

The plurality of electrodes 12 may be randomly and diffusely arranged on the base material 14. The plurality of electrodes 12 may also be arranged on the base material 14 at approximately equal intervals from each other. Although the electrode catheter 10 includes a plurality of the base materials 14, the arrangement patterns of the electrodes 12 arranged on each base material 14 may be all the same, or may be different on each or a part thereof.

Each electrode 12 is connected to a conducting wire 18. The conducting wire 18 extends from each electrode 12 to a root portion 20 of the base material 14 along the front surface or the back surface of the base material 14. In other words, a plurality of the conducting wires 18 are provided on the base material 14. All of the plurality of conducting wires 18 are aggregated into the root portion 20. Some of the plurality of conducting wires 18 extend along the front surface (corresponding to a first surface) of the base material 14, and the remaining others extend along the back surface (corresponding to a second surface) of the base material 14. The number of conducting wires 18 arranged on the front surface of the base material 14 and the number of conducting wires 18 arranged on the back surface of the base material 14 may be substantially the same. When the number of conducting wires 18 is odd, the number of conducting wires 18 arranged on one surface of the front surface or the back surface of the base material 14 may be required to be increased (or decreased) by one as compared with the other surface. The conducting wires 18 are not electrically connected to each other and are arranged so as not to overlap.

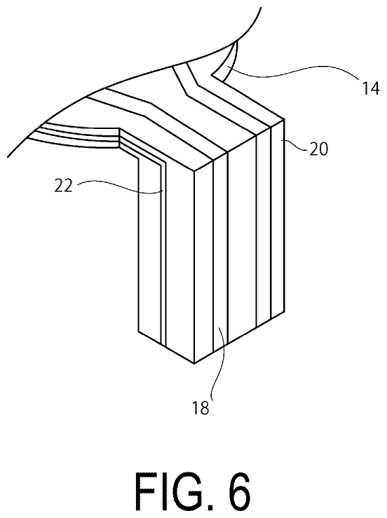

FIG. 6 is a perspective view illustrating an overview of the base material. Specifically, FIG. 6 is an enlarged view of the root portion 20 of the base material 14. As illustrated in FIG. 6, the root portion 20 is formed by a support extending along a direction (Z-axis direction) intersecting the main surface of the base material 14. In the illustrated example, the root portion 20 has a square cylinder shape, but is not limited thereto. Some of the plurality of conducting wires 18 (conducting wires 18 extending along the front surface) extend on one surface (−X side surface) of the root portion 20. The remaining others of the plurality of conducting wires 18 (conducting wires 18 extending along the back surface) extend on the other surface (+X side surface) of the root portion 20. That is, the plurality of conducting wires 18 extend along a surface (the surface of the root portion 20) that is continuous with the surface of the base material 14 on which the conducting wires 18 are arranged.

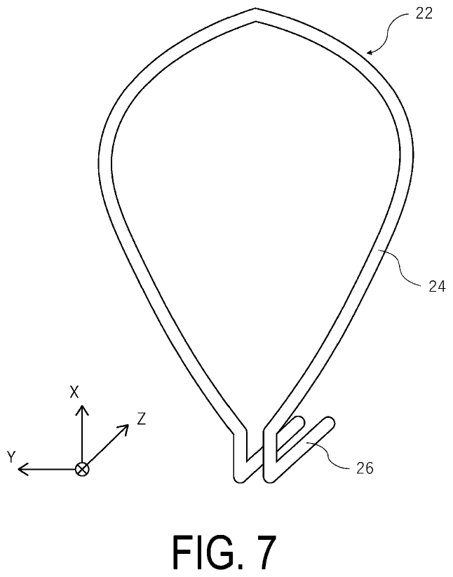

FIG. 7 is a perspective view illustrating a shape support structure. The shape support structure is constituted by a shape memory member 22 processed into a predetermined shape. The shape memory member 22 includes a first portion 24 extending along the periphery (around the Z-axis) of the base material 14 and a second portion 26 extending along the root portion 20. The first portion 24 and the second portion 26 are formed of one continuous wire rod. The shape memory member 22 is preferably formed of NiTi, for example. The first portion 24, like the base material 14, has an outline imitating a lanceolate leaf. The ends of the first portion 24 are spaced apart from each other. The second portion 26 is formed of two wire rods each extending along the Z-axis from the end of the first portion 24. The shape memory member 22 is joined along the periphery of the base material 14, and when no external force is applied to the shape memory member 22, the base material 14 is pulled and stretched (expanded) by the shape memory member 22. The shape memory member 22 is shaped so that the base material 14 can maintain the expanded state. Thus, the base material 14, in a state with no force acting, assumes a posture (expanded state) extending in the XY plane from the root portion 20.

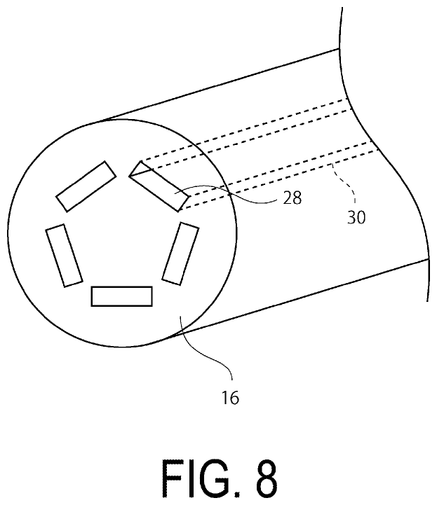

FIG. 8 is a schematic diagram illustrating the shaft. Specifically, FIG. 8 illustrates the vicinity of the end face of the shaft 16 in the +Z-axis direction. For clarity, only one lumen is illustrated in FIG. 8. A plurality of openings 28 are provided on the end face of the shaft 16. An opening 28 is a doorway to a lumen 30 that extends through the shaft 16. The plurality of lumens 30 each extends axially across the shaft 16 to the other end face. The root portion 20 of the base material 14 is inserted into each opening 28 and fixed by a fixing means such as an adhesive. One base material 14 is inserted into one opening 28. The conducting wires 18 of each base material 14 enter the shaft 16 through the opening 28 and exit shaft 16 through the opposite end face. Each lumen 30 is spatially independent of each other, and the plurality of conducting wires 18 are insulated between each other.

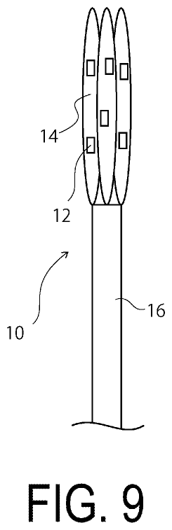

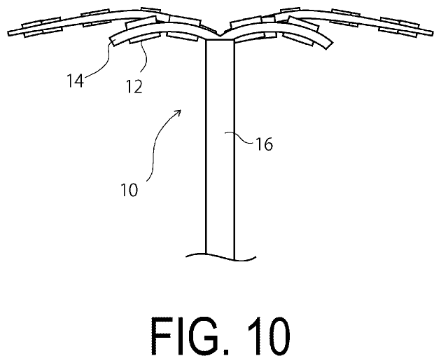

FIGS. 9 and 10 are side views of the electrode catheter. Specifically, FIG. 9 illustrates the electrode catheter 10 in a contracted state, and FIG. 10 illustrates the electrode catheter 10 in an expanded state.

As illustrated in FIG. 10, in the electrode catheter 10, the base material 14 extends radially outward in the expanded state. The plurality of base materials 14 are arranged so that the electrodes 12 do not overlap each other. In the transition between the expanded state and the contracted state, it is preferable that the base materials 14 do not overlap in order to make it difficult for the base materials 14 to interfere with each other. When no external force is acting on the electrode catheter 10, the restoring force of the shape memory member 22 keeps the electrode catheter 10 in the expanded state. When an external force of a predetermined value or more acts on the base material 14, the base material 14 deforms against the restoring force of the shape memory member 22. The base material 14 is deformable in both the front surface direction and the back surface direction. The base materials 14 may be deformed independently of each other.

As illustrated in FIG. 9, in the electrode catheter 10, all of the base materials 14 extend along the axis of the shaft 16 in the contracted state. That is, in the contracted state, all of the base materials 14 are aggregated along the axis of the shaft 16. As a result, the outer diameter of the electrode catheter 10 is entirely reduced. The electrode catheter 10 is contracted in a delivery sheath upon delivery.

Next, an operation of the electrode catheter 10 will be described.

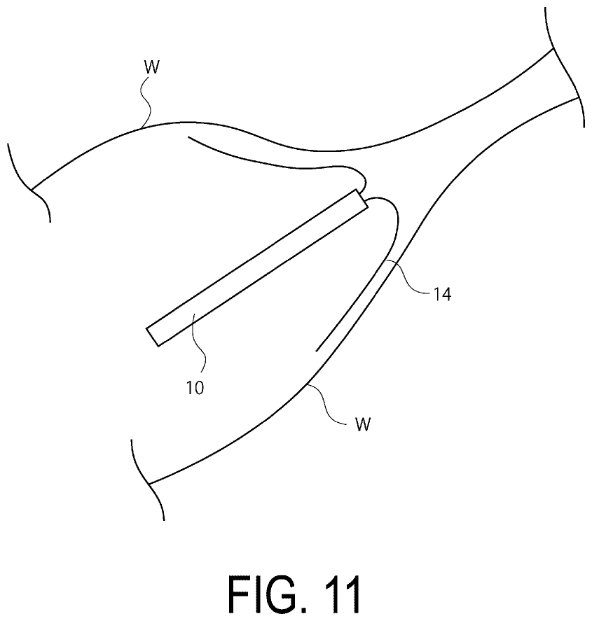

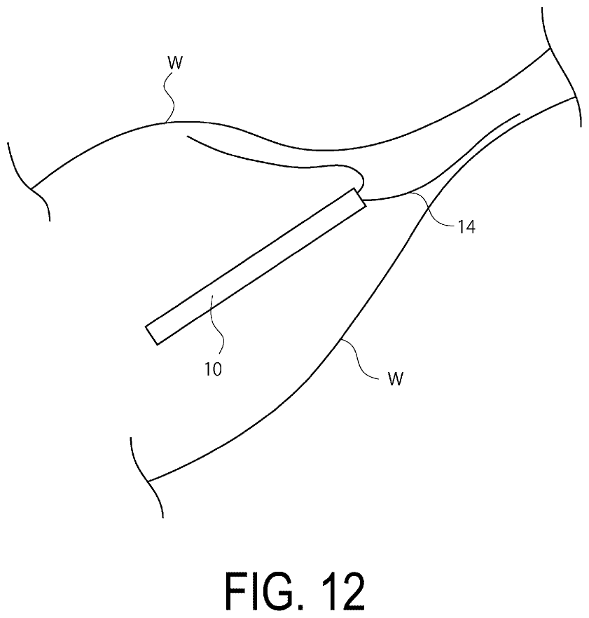

FIGS. 11 and 12 are diagrams illustrating usage states of the electrode catheter. FIGS. 11 and 12 illustrate a state in which a potential near the confluence point between the left atrium and the left pulmonary vein is mapped. In FIGS. 11 and 12, the electrode catheter 10 is in an expanded state. In FIG. 11, it can be seen that the surfaces (potential detection surface) of all base materials 14 of the electrode catheter 10 are in contact with a dissection wall W (potential detection target surface). On the other hand, in FIG. 12, the front surfaces of some of the base materials 14 are in contact with the dissection wall W, and the back surfaces of some of the base materials 14 are in contact with the dissection wall W.

When the electrode catheter 10 is moved from the contracted state to the expanded state, it may be difficult to control which face of the base material 14 contacts the dissection wall W, depending on the shape of the dissection wall W. On the other hand, since the electrode 12 is present on both sides of the base material 14, the electrode catheter 10 can perform mapping no matter which side of the base material 14 contacts the dissection wall W.

The embodiment is only one aspect of the present disclosure, and each configuration of the embodiment may be modified as appropriate without departing from the spirit of the present disclosure.

The tip of the base material 14 may be rounded in the XY plane or recessed to the root side. With this, the sharp shape of the tip of the base material 14 can be removed.

Adjacent base materials 14 may be connected to each other by a tensile member such as a tether. This can reduce the amount of deviation of the relative positions between each other of the base materials 14 in the circumferential direction.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. An electrode catheter, comprising:
a base material having a thin-film shape;
a shape support structure of the base material; and
a plurality of electrodes provided on the base material, wherein
at least some of the plurality of electrodes are exposed on a front surface and a back surface of the base material.

2. The electrode catheter according to claim 1, wherein
at least some of the plurality of electrodes are each connected to a conducting wire that extends along the front surface of the base material, and
a remainder of the plurality of electrodes is each connected to a conducting wire that extends along the back surface of the base material.

3. The electrode catheter according to claim 1, wherein the shape support structure is a shape memory member arranged along a periphery of the base material.

4. The electrode catheter according to claim 1, comprising:
a plurality of the base materials, wherein
the electrode catheter further comprises a shaft that holds a root of each of the plurality of the base materials; and
a connecting member that connects adjacent ones of the plurality of the base materials, and
the plurality of the base materials are arranged circumferentially around the shaft and are capable of assuming an expanded state and a contracted state.

5. The electrode catheter according to claim 4, wherein
at least some of the plurality of electrodes are each connected to a front-side conducting wire that extends along the front surface of the base material,
a remainder of the plurality of electrodes is each connected to a back-side conducting wire that extends along the back surface of the base material, and
the front-side conducting wire and the back-side conducting wire are axially provided on outer surfaces of a root along the root of the base material.

6. The electrode catheter according to claim 5, wherein
the root of the base material comprises a first surface facing a front side of the base material and a second surface facing a back side of the base material,
the front-side conducting wire is provided on the first surface, and
the back-side conducting wire is provided on the second surface.

7. The electrode catheter according to claim 1, comprising:
20 or more of the plurality of electrodes.

8. A potential detection device, comprising:
a base material having a thin-film shape;
a shape support structure of the base material; and
a plurality of electrodes arranged on the base material, wherein
at least some of the plurality of electrodes are exposed on a front surface and a back surface of the base material.

9. A potential detection method, comprising:
expanding a plurality of thin-film shaped base materials supported by a shape support structure in a contracted state; and
contacting a front surface of each of some of the plurality of thin-film shaped base materials with a potential detection target surface, and contacting a back surface of each of a remainder of the plurality of thin-film shaped base materials with the potential detection target surface.
